(19) United States
(12) Patent Application Publication
(10) Pub. No.: US 2004/0127870 A1
(43) Pub. Date: Jul. 1, 2004

(54) Thin curved elasticized absorbent article with absorbent concentration profile

(76) Inventors: Joseph DiPalma, Neenah, WI (US); Teresa M. Zander, Bonduel, WI (US)

Correspondence Address: GENERAL NUMBER 00757, BRINKS HOFER GILSON & LIONE, P.O. BOX 10395, CHICAGO, IL, 60610, US

(21) Appl. No.: 10/330,417

(22) Filed: Dec. 27, 2002

(51) Int. Cl. A61F 13/15; A61F 13/20

(52) U.S. Cl. 604/378

(57) ABSTRACT

An absorbent article with increased body-fit and leakage protection has a concentrated center region. This central region is concentrated in the center of the article allowing the surrounding regions to remain thin for increased wearer comfort. The concentrated central region also provides support for elastic side members that form the article to the wearer's body.

This invention relates to a thin absorbent article for absorbing and retaining human exudates. More specifically, this invention relates to thin sanitary napkins having an absorbent concentration profile. The article maintains an overall thickness and absorbent capacity that is desired by the consumer, and conforms to the wearer's body without compromising comfort.

### BACKGROUND

Absorbent articles such as sanitary napkins, incontinent garments, incontinent shields, and the like are designed to be worn adjacent to a woman's pudendum to absorb body fluids such and menses, blood, urine, and other excrements. Some of these articles are designed to be worn during the menstrual period, while others are intended to be worn before and after the menstrual period or during periods of incontinence. Today, thin absorbent articles, having an overall thickness of about five millimeters or less, are preferred by a large number of women.

In the past, absorbent articles have either been thick to absorb a heavy flow or thin to accommodate a lighter discharge. Due to consumer preference that the overall thickness of the absorbent article be as thin as practical to increase comfort and discretion, there is a need for a thin absorbent article that will absorb larger amounts of body fluid. One way to make a thin article that has increased absorbency is to concentrate the absorbent material in the center of the article, as opposed to the surrounding absorbent region. Having an article with a thin surrounding absorbent region is desired by many consumers and a concentrated center does not interfere with the benefits of the thin article. However, while adding absorbent material to the center of a thin article does increase its absorbent capability, it does not allow the article to conform to the wearer's body. This deficiency causes fluid leakage because the article shifts away from the wearer's body. Also, the thin surrounding absorbent region of the article allows fluid that is not quickly absorbed to leak from the sides of the article.

One proposed solution is to provide a thinner absorbent article that includes elastic sides. This would provide better leakage protection around the inner thighs as well as conforming the article to the shape of the wearer's body. However, when elastic is attached to a traditional thin absorbent article, the article doubles over, longitudinally, onto itself. To solve this problem, it has been proposed to make the article stiff or to provide ribs throughout the sides of the article. Both proposed solutions compromise the wearer's comfort.

Accordingly, there is a need for thin absorbent articles with increased absorbency. Furthermore, the article must comfortably conform to the wearer's body to prevent fluid leakage. The present invention is an absorbent article that will remedy this, and other, problems of the prior endeavors. These attributes will become clear as the present invention is more thoroughly discussed in this application.

### SUMMARY

The present invention is directed to an absorbent article that is both thin, to accommodate the comfort of the wearer, and contains elastic sides, which conforms the absorbent article to the wearer's body. The thin elasticized absorbent article maintains its shape while having a surrounding absorbent region that is no more than five millimeters thick. The article concentrates the absorbent core of the article in such a manner that the core has a concentration profile of about 13 to about 24. Centralized distribution of the absorbent mass allows the center of the article to rise to the wearer's body to readily accept fluid, to generate a drier body-facing surface by providing a greater depth for desorbtion to occur, to resist deformation and retain its shape at the centermost portion that is subject to the highest forces, and to maintain overall absorbent capacity in the product while freeing up the surrounding absorbent regions to conform to the natural curves of the body.

An absorbent article having features of the present invention includes a liquid permeable cover layer, a liquid impermeable baffle layer, and an absorbent core, which is enclosed within the cover and baffle layers. The present invention also includes elastic side members that are disposed within the side peripheral seal of the cover and baffle layers.

The absorbent core is comprised of at least two discrete absorbent components, a first absorbent layer and an absorbent pledget. The absorbent core has a concentration profile of about 13 to about 24, and desirably, at least about 14, and more desirably at least about 15. The concentration profile of the core is determined by calculating the mass proportion ratio of the discrete absorbent components, as described in more detail below.

The first component of the absorbent core, the first absorbent layer, is positioned between the cover and baffle layers. The absorbent layer may be in the shape of a dog bone or an hour glass, having two rounded end portions and a narrower central portion, or a race track. This layer may be about 60% to about 99% of the length of the absorbent article, itself, and more desirably at least about 80% of the entire absorbent article's length.

The second absorbent component of the core, the absorbent pledget, is generally positioned below the first absorbent layer and above the baffle layer when the article is positioned on the wearer. The absorbent pledget may be oval, rectangular, or elliptical in shape with two opposing side edges, a front edge, and a rear edge, such that all of the pledget's edges are located within the outer edges of the first absorbent layer. The length of the absorbent pledget is approximately one third the length of the first absorbent layer. Desirably, the absorbent pledget is located in approximately the center portion of the article. The pledget, however, may be placed toward the front portion or the rear portion of the first absorbent layer.

Thin absorbent articles according to the present invention generally have a thickness of about 2 mm to about 8 mm, desirably from about 3 mm to 5 mm. The cover layer, baffle layer, and absorbent layer should each be about 0.1 mm to about 4 mm thick. Their total thickness should be no more than about 5 mm. In addition, the area surrounding the absorbent pledget has a basis weight (BWₛ) of 130-400 g/m², more desirably 150-250 g/m². This basis weight allows the concentration profile of the absorbent core to be such that it will prevent the longitudinal collapse of the article. In other words, the area surrounding the absorbent pledget must have more strength than a light-weight tissue-like material.

The elastic side members are generally disposed in the peripheral seal formed at the periphery of the cover and baffle layers. The elastic members are disposed about the opposed sides of the article and are approximately the same length as the absorbent pledget. The elastic members may be composed of any suitable elastic material.

In other embodiments, the absorbent core may be comprised of more than two discrete layers. For example, in one embodiment, the absorbent core may include a first absorbent layer, an absorbent pledget, and an outer ring. In this embodiment, the outer ring is disposed between the absorbent pledget and the baffle layer. The ring will have a thickness of about 0.1 mm to about 4 mm and will provide structural support for the absorbent core. The total thickness of the cover layer, the baffle layer, the first absorbent layer, and the outer ring, will remain, at most, about 5 mm, despite the addition of the ring layer. Optionally, a fluid impermeable barrier layer may be disposed between the absorbent pledget and the ring layer.

In another embodiment, the absorbent core includes two absorbent layers and an absorbent pledget. The two absorbent layers each have lengths that range from about 60% to about 99% of the length of the entire absorbent article. Desirably, the two absorbent layers each have lengths that are at least 80% the length of the entire absorbent article. The pledget may disposed between the two absorbent layers. The three discrete layers of the absorbent core may be made of an airlaid or fluff material.

As in the embodiment including the ring layer, the added absorbent layer will have a thickness of about 0.1 mm to about 4 mm and the total thickness of the layers, not including the absorbent pledget, is no more than about 5 mm.

Generally, all of the described embodiments may include an intake layer. Personal care absorbent articles, such as sanitary napkins, are often required to accept quick, large insults of body exudates that are beyond the immediate absorptive capacity of the product. As a result, it has been found advantageous to use intake layers within such absorbent articles. Generally, when an intake layer is present, it is disposed between the cover layer and the absorbent core. In addition, it is helpful if the intake layer is attached to the cover layer and the absorbent core to promote liquid transfer.

The present invention also includes a method for preventing the longitudinal collapse of an ultrathin absorbent article fitted with elastic side members comprising providing an absorbent core that has concentration profile of about 13 to about 24. Moreover, the support for the absorbent core is provided by use of an absorbent pledget that has a greater concentration of absorbent material than does the region surrounding the pledget.

### BRIEF DESCRIPTION OF THE DRAWINGS

These and other features, aspects, and advantages of the present invention will be better understood with regard to the following description, claims, and accompanying drawings where:

FIG. 1 is a perspective view of an embodiment of the present invention.

FIG. 2 is a top view of the article of FIG. 1.

FIG. 3 is a perspective view of another embodiment of the present invention with optional side flaps.

FIG. 4 is a cross-sectional view of the article of FIG. 2 along line 4-4.

FIG. 5 is an exploded view of the article of FIG. 1.

FIG. 6 is a perspective view of another embodiment of the present invention.

FIG. 7 is a top view of the article of FIG. 6.

FIG. 8 is a cross sectional view of the article of FIG. 7 along line 8-8 with the optional barrier layer disposed therein.

FIG. 9 is an exploded view of the article of FIG. 6 with the optional barrier layer disposed therein.

FIG. 10 is a perspective view of another embodiment of the present invention.

FIG. 11 is a top view of the article of FIG. 10.

FIG. 12 is a cross sectional view of the article of FIG. 11 along line 12-12.

FIG. 13 is an exploded view the article of FIG. 10.

### DESCRIPTION

### Definitions

“Airlaid” refers to a process for making material wherein fibers, such as cellulose-type fibers are arranged on a wire as a base sheet, and where the base sheet can be sprayed with an adhesive or combined with heat-activated fibers, powders, or the like. Alternatively, the base sheet can be calendered with sufficient heat and pressure to produce significant hydrogen bonding between base sheet components. The airlaid material is thus a bonded material. An example of such material is disclosed at U.S. Pat. No. 4,100,324, the relevant portions of which are incorporated herein by reference. A commercially available airlaid fabric is sold by Concert Industries Ltd. Located at 350 rue Nash, Thurso, Quebec, Canada, JOX 3B0. This airlaid is 90% virgin sorftwood held together by a bicomponent fiber with a polypropylene core and a polyethylene sheath, type #T255 fiber available from KoSa, P.O. Box 4, Highway 70 West, Salisbury, N.C., 28145.

The term “apertured film” refers to a liquid permeable material that contains a plurality of holes.

The term “Concentration Profile” (CP), is calculated by dividing the mass proportion of the center absorbent region, (MP_{c}), by the mass proportion of the surrounding absorbent region, (MPₛ). More specifically, the mass proportion of center absorbent region (MP_{c}) is calculated by dividing the basis weight of the center absorbent region (BW_{c}) by the area of the center absorbent region (A_{c}). Likewise, the mass proportion of the surrounding absorbent region (MPₛ) is calculated by dividing the basis weight of the surrounding absorbent region (BWₛ) by the area of the surrounding absorbent region (Aₛ). The basis weights are calculated by dividing the weight of the respective regions by their areas.

CP=MP_{c}/MPₛ=(BW_{c}/A_{c})/(BWₛ/Aₛ)

As used herein, the “crotch region” of an absorbent article refers to the generally central region that will be in contact with the crotch of a user, near the lowermost part of the torso, and resides between the front and rear portions of the article. Typically, the crotch region generally spans approximately 7 to 10 cm in the longitudinal direction.

The term “pledget” refers to a small compressed layer of absorbent material.

The term “super absorbents” refers to a water-swellable, water-insoluble, organic or inorganic material capable, under the most favorable conditions, of absorbing at least about 15 times its weight and, more desirably, at least about 30 times its weight in an aqueous solution containing 0.9 weight percent sodium chloride. The super absorbent materials can be natural, synthetic, and modified natural polymers and materials. In addition, the super absorbent materials can be inorganic materials, such as silica gels, or organic compounds such as cross-linked polymers. An example of super absorbents is disclosed in U.S. Pat. No. 5,609,588, column 8, line 1 to line 57, which portion is incorporated herein by reference.

“Tensile strength” refers to the resistance of a material to a force tending to tear it apart. The strength is measured as the maximum tension the material can withstand without tearing.

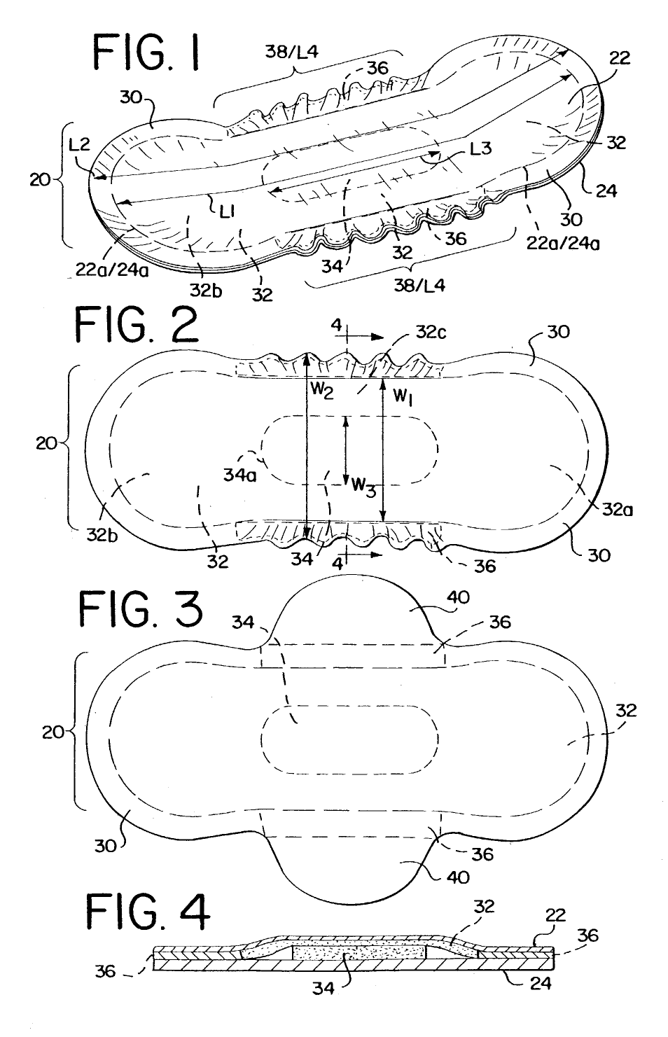

Turning now to FIGS. 1-5, a thin absorbent article 20 is shown that is capable of absorbing body fluid. The absorbent article 20 can be a diaper, training pant, sanitary napkin, a panty liner, an overnight pad, an incontinent garment, or any other known disposable absorbent product capable of absorbing urine, menses, blood, perspiration, excrements or other bodily fluids discharged by a human. The absorbent article 20 is designed to be secured to an inside surface of a person's undergarment by a garment attachment adhesive.

The article 20 includes a fluid permeable cover layer 22, a baffle layer 24, and an absorbent core 26. The absorbent core 26 is disposed between the cover layer 22 and the baffle layer 24 and has a concentration profile of about 13 to about 24, desirably at least about 14, and more desirably at least about 15. The absorbent core 26 includes at least two discrete components: an absorbent layer 32 and an absorbent pledget 34. The total thickness of the cover layer 22, the baffle layer 24, and the absorbent layer 32 is about 2 mm to about 5 mm. In addition, elastic side members 36 are disposed between the cover layer 22 and the baffle layer 24 in at least a portion of opposing side edges 38 of the article 20.

The article 20 has a length L₂that is about 120 mm to about 320 mm, and a width W₂ of about 60 mm to about 180 mm. The article 20 may have any suitable shape. For example, the article may have a dog bone shape with rounded ends. One skilled in the art would understand that the shape of the absorbent article is not a limiting factor. Using the cover layer 22 as a point of reference, the components of the absorbent core 26 are arranged so that the first absorbent layer 32 is positioned adjacent to the cover 22, the absorbent pledget 34 is positioned below the first absorbent layer 32 and adjacent the baffle layer 24.

Looking at some of the elements of the absorbent article 20 more specifically, the cover layer 22 is designed to contact the body of the user and desirably is liquid-permeable. The cover layer 22 can be constructed of a woven or nonwoven material, from synthetic or natural materials and should be easily penetrated by body fluid. Suitable materials include bonded carded webs of polyester, polypropylene, polyethylene, nylon or other heat-bondable fibers. Other polyolefins, such as copolymers of polypropylene and polyethylene, linear low-density polyethylene, finely perforated film webs and net material also work well. A preferred cover material is a 0.6 ounces per square yard (osy) spunbond polypropylene. Spunbond and bonded carded webs are commercially available from Kimberly-Clark Corporation having an office at 401 N. Lake Street, Neenah, Wis., 54956. Another nonwoven material that can be used as the cover layer 22 is formed from 100 percent polyester fibers held together by a binder. This material is known as powder bonded carded web (PBCW). PBCW is commercially available from HDK Industries, Inc. having an office at 304 Arcadia Drive, Greenville, S.C. 29609. The cover layer 22 can also be constructed of a thermoplastic film that contains apertures 28 and is flanked on both sides by a nonwoven material. This particular material provides a soft feel against a user's thigh while allowing body fluid to rapidly pass therethrough.

In order to facilitate movement of body fluid down in the absorbent article 20, it is possible to form a plurality of apertures 28 in the cover layer 22. The apertures 28 can be randomly or uniformly arranged throughout the cover layer 22. The apertures 28 permit rapid penetration of body fluid down into the absorbent core 26. The size, shape, and diameter, of any number of apertures 28 can be varied to suit one's particular needs. In addition, the cover layer 22 can be treated with a surfactant to make it more hydrophilic. By “hydrophilic” it is meant that the cover layer 22 will have a strong affinity for water. The cover layer 22 can also be embossed to improve the aesthetic appearance of the absorbent article 20.

The baffle layer 24 is generally liquid-impermeable and is designed to face the inner surface, i.e. the crotch portion, of an undergarment (not shown). The baffle 24 can be designed to permit a passage of air or vapor out of the absorbent article 20 while blocking the passage of liquids. The baffle 24 can be made from any material having the above identified properties. A good material is microembossed, polymeric film, such as polyethylene or polypropylene. One material that may be used is a polyethylene film having a thickness in the range of about 0.2 mm to about 5.0 mm and desirably about 0.5 mm to about 3.0 mm.

The cover 22 and baffle 24 can be coextensive in a face-to-face contact around the outer edge of the absorbent core 26. The cover 22 and baffle 24 can be sealed together about their peripheries 22a and 24a by use of an adhesive, by heat sealing ultrasonics, or by any other process known in the art. This sealed area is the peripheral seal 30. The peripheral seal 30 has end margins 30a and side margins 30b. The length and width dimensions of the cover 22 and baffle layer 24 are generally larger than and extend beyond the corresponding dimensions of the absorbent core 26 to provide for the corresponding end margins 30a and side margins 30b. In addition, the cover layer 22 and baffle layer 24 can have a thickness between about 0.1 mm to about 4 mm, desirably less than about 1 mm. The shape, length, and width, of the absorbent article 20 will be defined by the peripheral edges of the cover and baffle layers.

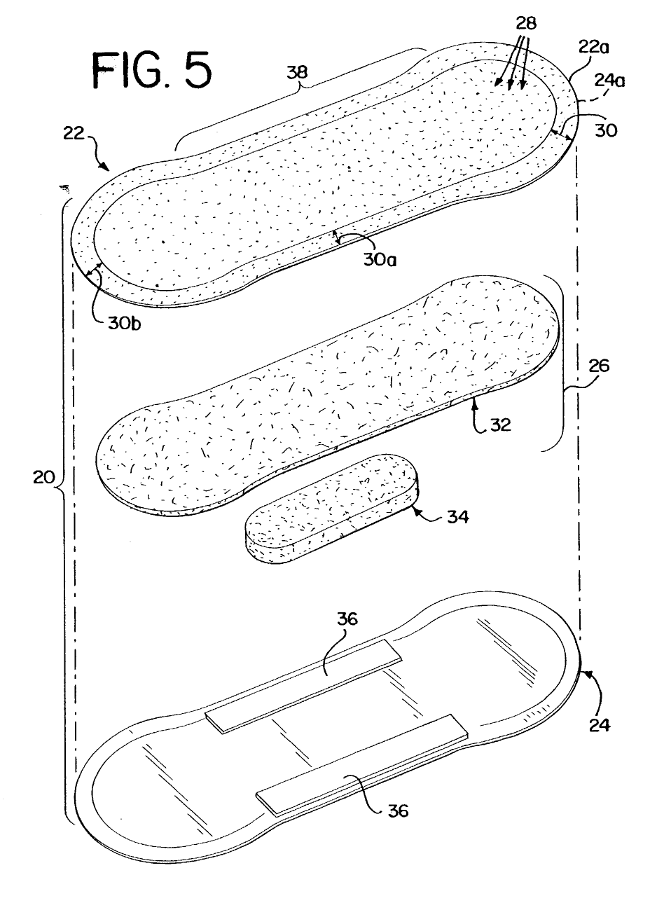

Referring now to FIG. 4 and FIG. 5, the absorbent article 20 of the present invention includes an absorbent core 26. The absorbent core 26 provides an absorbent structure that is configured for holding and storing absorbed liquids and other waste materials. The core 26 can be positioned and sandwiched between the cover layer 22 and the baffle layer 24 to form the absorbent article 20. Desirably, the absorbent core 26 is enclosed between the cover 22 and baffle layers 24 by the peripheral seal 30. The absorbent core 26 is comprised of at least two discrete absorbent components: a first absorbent layer 32 and an absorbent pledget 34. The core 26 has a concentration profile of about 13 to about 22, desirably at least about 14, and more desirably at least about 15. In addition, the basis weight ratio of the core (BW_{c}/BWₛ) is at least about 3.

The absorbent core 26, and any of its layers, can be made of any material that will absorb bodily exudates such as menses, blood, catamenial fluids, and urine. Suitable absorbents include cellulose fluff, wood fluff, rayon, cotton, and airlaid. Meltblown polymers, such as polyester, polypropylene, with staple cellulose fibers, can also be used. Desirably, a stabilized absorbent having a binder or a fluff material could be used for the first absorbent layer 32, more desirably an airlaid material is used. Superabsorbent materials may be used or odor control ingredients, such as activated carbon, may be added to the first 32 and/or second 44 absorbent layer or the absorbent pledget 34.

The first component of the absorbent core 26, the absorbent layer(s) 32, may have any suitable shape. One desired shape is an hour-glass or bar bell shape with two enlarged end portions 32a and 32b. The end portions 32a and 32b are relatively flat with rounded profiles that are separated by a narrower center portion 32c. The length and width dimensions of the absorbent layer 32 are less than the length and width dimensions of the cover 22 and baffle layers 24. The absorbent layer(s) 32 may have a length L₁ that is at least about 80% of the length L₂ of the absorbent article 20 itself. Moreover, the absorbent layer 32 has a thickness of about 0.1 mm to about 4.0 mm. Together, the total thickness of the cover layer 22, the baffle layer 24, and the absorbent layer 32, is about 2 mm to about 8 mm, desirably about 3 mm to about 5 mm.

The absorbent layer 32 can be configured to have increased tensile strength characteristics in comparison to many conventional absorbent structures, thereby presenting improved processing capabilities. In particular, improved wet tensile strength allows the absorbent layer 32 to better maintain the shape of the article and prevent the article from longitudinally doubling over, onto itself.

The second component of the absorbent core 26, the absorbent pledget 34, will be more absorbent than the other components. It is desirable to have a pledget 34 that is about 3 to about 8 mm thick. It may be composed of any suitable material for absorbing body fluids. Desirably, the absorbent pledget 34 will be an airlaid or fluff material. The pledget 34 may have any suitable shape. Some desired shapes are oval, elliptical, or rectangular.

Moreover, the absorbent pledget 34 may be positioned in the center third portion of the length of the absorbent article L₂, between the absorbent layer(s) 32 and the baffle layer 24. The absorbent pledget 34 has a length L₃ that is approximately one-third the length of the absorbent layer(s) L₁ and a width W₃ that is less than the width W₁ of the absorbent layer 32. The length of the pledget L₃ (one-third of the length of the absorbent layer L₁) corresponds to the general length of the elastic side members L₄. Making the pledget 34 and the elastic side members 36 of corresponding lengths creates good body fit and allows the article 20 to conform well to the wearer's body. Additionally, the one-third measurement allows one to easily tri-fold the absorbent article for packaging.

The area surrounding the absorbent pledget, the surrounding absorbent region, has a basis weight (BWₛ) of about 130 to about 400 g/m², more desirably from about 150 to about 250 g/m². This basis weight, along with the concentration profile from about 13 to about 24, creates a thin article that will resist the tension created by the elastic side members. The article 20 need not be stiffened by any other methods, when combined with elastic side members 36, to keep the article 20 from doubling over, longitudinally, onto itself.

The first absorbent layer 32 and the absorbent pledget 34 may be attached to one another using several different methods known to those skilled in the art. The attachment methods desirably maintain a secure surface interface between the absorbent portions and allow for the maximum of fluid transfer between the absorbent layers. Generally an adhesive is sprayed on either the first absorbent layer 32 or the absorbent pledget 34 in order to attach the two components.

The absorbent article 20 also comprises elastic side members 36. The elastic side members 36 are located in the side margins 30b of the absorbent article 20 between the cover layer 22 and the baffle layer 24. The elastic side members 36 may extend beyond the outer side edges of the first absorbent layer 32. Also, the elastic side members 36 may be applied to the garment-facing side of the baffle layer 24 at the side margins 30b. In this embodiment, the cover layer 22 is created to have a greater width than the width of the baffle layer. The cover layer 22 can then be folded over the garment-facing side of the baffle layer 24 to cover the elastic side members 36.

The elastic side members 36 are arranged to draw and hold the absorbent article 20 against the legs and body of the wearer. The elastic side members 36 are secured to the absorbent article 20 in an elastically contractible condition so that in a normal under strain configuration, the elastic members effectively contract against the article. Desirably, the length of the elastic side members L₄ is approximately equal to the length of the central absorbent pledget L₃.

The elastic side members 36 may be made of any suitable elastic material and can be in the form of a single, relatively flat rectangular strip, a single strand of elastic, or a plurality of individual elastic strands. In one embodiment a Lycra® material was used and available from DuPont Apparel & Textile Sciences, Chestnut Run Plaza, Building 728, 4417 Lancaster Pike, Wilmington, Del., 19805. The cross-sectional shape of the elastic strands can vary and may include round, oval, square, rectangular or irregular shapes. Heat shrinkable elastics can be used and offer the advantage in that the elastic may be applied as a nonelastic film which becomes elastic upon heating. Heat-shrinkable elastics are also relatively easy to form into particular shapes by controlling the method and location of the heat source. For example, the elastic may be applied in a rectangular strip form and heat can be applied from the cover layer-side surface of the article 20 such that the absorbent will insulate the heat-shrinkable elastic from the applied heat and permit only the area in the exposed walls to shrink. Exemplary heat shrinkable elastics are those taught in U.S. Pat. Nos. 3,912,565, 3,819,401, and 3,639,917, the relevant portions of which are incorporated herein by reference.

When non heat-shrinkable elastics are utilized, the elastic can be applied as elongated strips that are secured in place, for example by being sewn or by use of an adhesive, while under tension and then allowed to relax. Such materials and processes for their application are well known in the art.

Referring now to FIG. 3, the absorbent article 20 may further comprise side flaps or wings 40. The side flaps 40 lie in at least the absorbent article's opposed side edges 38 and extend laterally outward beyond the side edges 38. The flaps 40 may be folded about the leg elastics of a wearer's panties and joined to the outside surface of the panties. A pressure sensitive adhesive (not shown) may be disposed on the garment surface of flaps 40 adjacent the distal edge thereof (i.e. the surface which contacts the outside surface of he wearer's panties when the flaps are folded back) to separably join the flaps 40 to the wearer's panties. The pressure sensitive adhesive may be covered with a silicon-coated paper (not shown) prior to use.

Each flap 40 may be formed integral with and as an extension of the cover layer 22 and the baffle layer 24 (not shown). The components of each flap 40 may be joined to each other by adhesive. Alternatively, the flaps 40 can include separate elements that are joined to one of the cover layer 22, the baffle layer 24, or both the cover layer 22 and the baffle layer 24 using methods known to those skilled in the art such as ultrasonic welding, adhesive bonding, or other means known to the art. Each flap 40 may be made of any suitable material. For example, flaps 40 may comprise the same material as used for the cover layer 22 or the same material as used for the baffle layer 24.

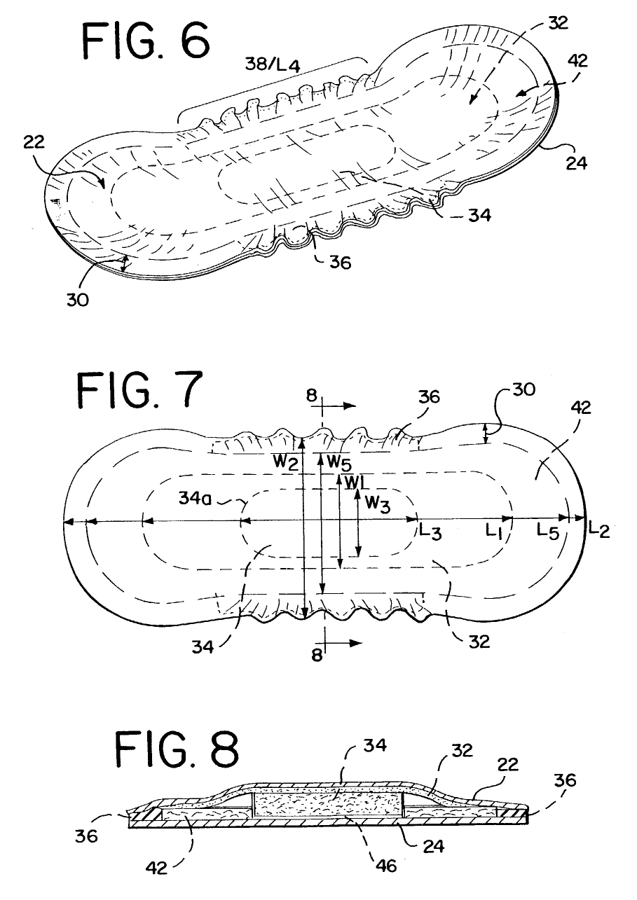

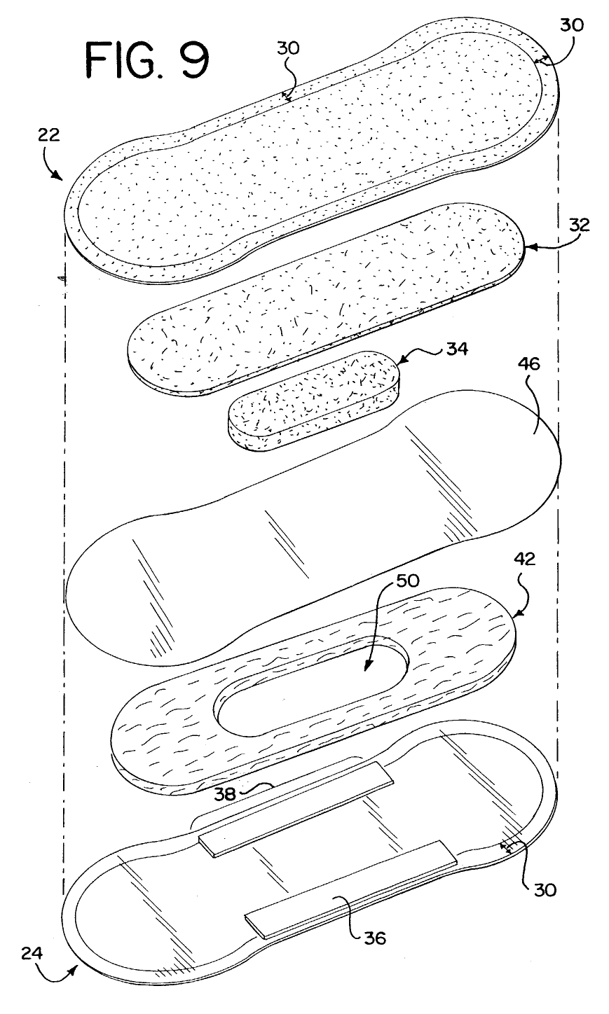

Another embodiment may have an absorbent core 26 that includes more than two layers. Referring now to FIGS. 6-9, wherein like reference numerals refer to like materials, the absorbent core 26 of the invention may be comprised of an absorbent layer 32, an outer ring 42, and an absorbent pledget 34, and a liquid impermeable barrier 46.

The outer ring 42 may be adjacent to the outer edges of the absorbent pledget 34a or the ring and the pledget may be separated by a liquid impermeable barrier 46. The outer ring 42 has a width W₅ and a length L₅ that is greater than the width W₃ and length L₃ of the absorbent pledget and a length L₅ and a width W₅ may be greater than, equal to, or less than, the width W₁ and length L₁ of the absorbent layer. Desirably, the length L₅ and width W₅ of the outer ring 42 is greater than the width W₁ and length L₁ of the absorbent layer 32.

The outer ring 42 can comprise a central cavity 50 passing completely through the layer to receive the central absorbent pledget 34. The outer ring 42 can be fluff material. The fluff material is desirable because the excess may be recycled and reused once the cavity is cut from the outer ring 42. The outer ring layer 42 can also be a composite element, such as a layer of cellulosic fibers joined to a polymeric foam layer. In one embodiment, the outer ring 42 is extensible such that its size can be adjusted for improved fit.

Desirably, the outer ring's 42 primary function is not to absorb fluid but to provide overall shaping, comfort, and integrity. The outer ring 42 can be porous, such as a ring of polyurethane foam; a polyethylene foam such as the product known as VOLARA™ 2a polyethylene foam, obtained from Voltek Corp., of Lawrence, Mass.; or a foam rubber material (e.g., foamed styrene butadiene), foamed silicones, or foamed vinyl plastics. Several such foams can be obtained from Woodbridge Foam Fabricating, Inc., located in Chattanooga, Tenn., from the E. N. Murray Company, Inc., located in Denver, Colo., and Astro-Valcour, Inc., located in Glens Falls, N.Y. Foam materials desirably have a density of about 0.02 grams per cubic centimeter (g/cc) to about 0.1 g/cc. The foam material may be treated to be absorbent and/or hydrophilic, but need not be hydrophilic.

A fluid impermeable barrier 46 may be disposed between the absorbent pledget 34 and the outer ring layer 42. The barrier 46 can comprise material from the baffle layer 24 of the absorbent article 20 and can be embossed, deformed, pleated, or stretched.

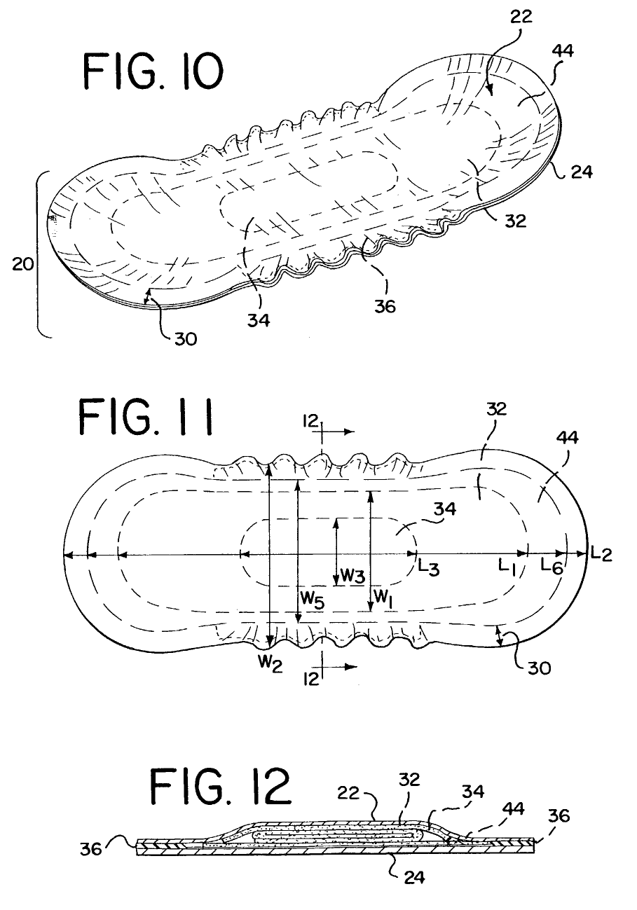

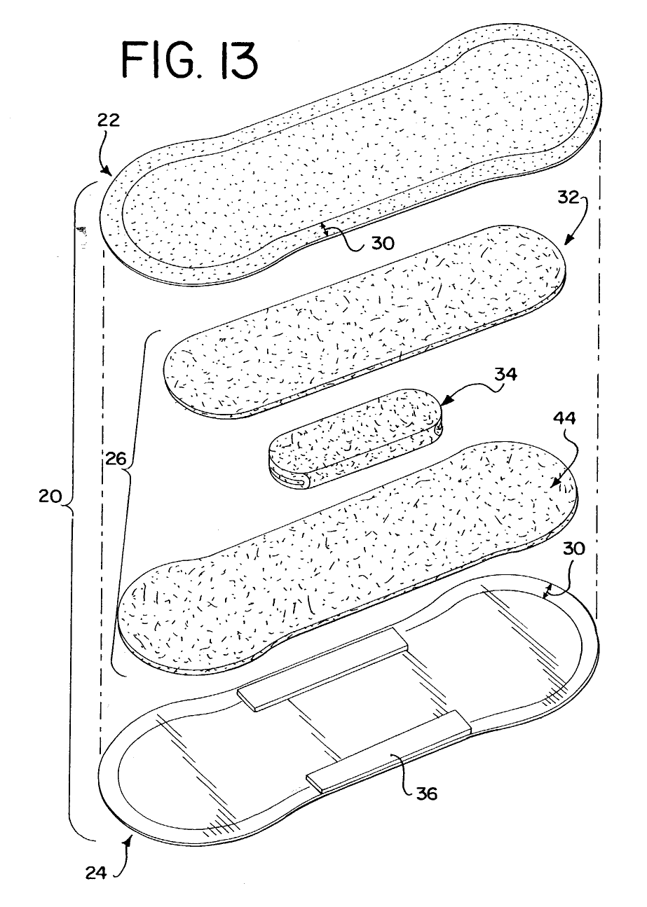

Referring now to FIGS. 10-13, another embodiment of the present invention is shown wherein like reference numerals refer to like materials. The absorbent core 26 is comprised of three layers: a first absorbent layer 32, a second absorbent layer 44, and an absorbent pledget 34. The first absorbent layer 32 and the second absorbent layer 44 are disposed between the liquid permeable cover layer 22 and the liquid impermeable baffle layer 24. The first absorbent layer 32 and the second absorbent layer 44 have a length L₁ and L₆ that is about 60% to about 99% of the length of the absorbent article L₂, and more desirably about 80% to about 90% of the article's length L₂.

The absorbent pledget 34 is disposed between the first absorbent layer 32 and the second absorbent layer 44. The absorbent pledget has a length L₃ that is approximately one-third the length of the absorbent article L₂ and the width of the absorbent pledget W₃ is less than the width of the first and second absorbent layers W₁ and W₆. The three absorbent layers may be comprised of an airlaid material, as defined above. The absorbent pledget 34 may be folded in the shape of the letter “c”, “v”, “z”, or “e”, desirably letter “e”, and be composed of an airlaid material.

Optionally, an intake layer (not shown) may be added to any one of the above embodiments. An intake layer provides added protection against increased liquid flow. In addition, an intake layer will help to maximize the absorbent properties of the absorbent article 20. The material to be used as an intake layer may be disposed between the cover layer 22 and the absorbent core 26. When an intake layer is present, it is helpful if it is attached to the cover layer 22 and the absorbent core 26 to promote liquid transfer. Examples of materials that may be used as an intake layer can be found at U.S. Pat. No. 5,486,166, the relevant portions of which are incorporated herein by reference.

Other components may be combined with the cellulosic materials of the absorbent core 26 or added as separate components, layers, or portions of the article. Such other components include odor absorbing components such as baking soda, talc powder, cyclodextrin, ethylenediamine tetra-acetic acid or other chelating agents, zeolites, activated silica, and activated carbon granules, fabrics or fibers; superabsorbent particles and fibers; antimicrobial agents including the silver-loaded zeolites of BF Technologies, located in Beverly, Mass., sold under the trademark HEALTHSHIELD™, as well as triclosan products, chitosan or chitin derivatives; polycarboxylic acids; encapsulated perfumes; emollients such as lanolin; or skin wellness agents such as aloe vera extract, emu oil, avocado oil, grape seed extractives, or vitamin E. Thermoplastic binder fibers may be added, with or without subsequent heat treatment for improved stability. Foam layers, foam shape-defining components, or foam particles may also be present. Plastic inserts to define shape or maintain integrity may also be used.

This invention also comprises a method for making a thin absorbent article with elastic side members. The method includes providing a structure to support the article to counteract the tension created by the elastic side members. Generally, the method includes using a discrete central absorbent layer or pledget that is equal in length to that of the elastic side members. The absorbent material is concentrated in the center of the absorbent core to allow the surrounding absorbent region of the article to remain thin and increase the comfort for the wearer. The absorbent pledget provides support for the elastic side members, and keeps the absorbent article from collapsing longitudinally.

This invention also includes an elasticized absorbent article that uses a concentrated, centrally located, pledget to counteract the tension created by the elasticized side member. When elastic is added to a thin absorbent article, the article collapses, longitudinally, because of the elastic tension. The present invention comfortably resists the elastic tension and gently curves the article to conform to the wearer's body by providing a region surrounding the absorbent pledget with a basis weight (BWₛ) of at least about 130-400 g/m², more desirably 150-250 g/m², and an absorbent core with an overall concentration profile of about 13 to about 24, desirably at least about 14, and more desirably at least about 15.

### EXAMPLES

In order to calculate the concentration profiles for the various articles, the mass proportion of the center region (MP_{c}) is divided by the mass proportion (MPₛ) of the surrounding absorbent region. The following equation was used to calculate the respective concentration profile (CP):

CP=MP_{c}/MPₛ=(BW_{c}/A_{c})/(BWₛ/Aₛ)=(BW_{c}/BWₛ)×(Aₛ/A_{c}).

To determine each of the above values, the following values are determined.

Total Absorbent Area (Aₜ)

First, the length (X) and the width (Y) of a sheet of paper having a size greater than the size of the absorbent article are measured in millimeters. Using the length (X) and width (Y), the area of the sheet (Aₚ) is calculated using the following equation:

Aₚ=X*Y/1000².

Then, the weight of the sheet of paper is measured (Wₚ). After the weight of the paper (Wₚ) has been measured, the portion of the absorbent article to be tested is placed on the sheet of paper and its peripheral shape is traced onto the paper. The traced shape of the absorbent is cut from the paper and its weight is measured (Wₜ). The area of the shape of the absorbent (Aₜ) is determined using the following equation:

Aₜ=Aₚ*(Wₜ/Wₚ).

Center Absorbent Region

First, an oval-shaped portion of each core, 32 mm wide by 80 mm long, is removed using a precision die cut. The precision die cutter used is available from Fox Valley Rule Die, Inc, 850 Declaration Dr., Neehnah, Wis., 45956. The area removed (A_{c}) is equal to 0.00256 m². The area removed (A_{c}) is carefully separated from the cover and baffle layer and is weighed (W_{c}) in grams. The basis weight (BW_{c}) is calculated using the following equation:

BW_{c}=W_{c}/A_{c}.

The oval to be cut from the central absorbent region is cut from the absorbent core at its heaviest point.

Surrounding Absorbent Region

The area of the surrounding absorbent (Aₛ) is equal to the total absorbent area (Aₜ) minus the center absorbent area (A_{c}). The area of the surrounding absorbent (Aₛ) is carefully separated from the cover and baffle layer and weighed (Wₛ) in grams. The basis weight of the area of the surrounding absorbent (Aₛ) is calculated using the following equation:

BWₛ=Wₛ/Aₛ.

Basis Weight Ratio

The basis weight ratio of the article's absorbent core is calculated by dividing the basis weight of the center (BW_{c}) by the basis weight of the surrounding (BWₛ).

Table I includes the calculations of the concentration profiles of various absorbent articles that were currently commercially available, at the time of the testing, and articles according to the present invention. The following examples are presented to more fully describe the present invention and should not be interpreted as limiting the invention in any way.

### EXAMPLE 1

The construction of an article according to the present invention is described from the top (or body-side) of the article, progressing downwards. A light amount of hot melt construction adhesive is sprayed beneath the 0.6 oz/yd² spunbond nonwoven cover layer. A first absorbent layer of 175 gm/m² stabilized airlaid with 90% cellulose and 10% binder fiber is cut to an hourglass shape and placed onto the interior of the cover layer. The density of this airlaid layer is 0.1 gm/cc. This layer is 66 mm wide at the center and 76 mm wide at the lobes and 218 mm long. An absorbent pledget of 100% unstabilized fiberized fluff at 650 gm/m² is cut to 36 mm wide by 82 mm long racetrack shape and located beneath the center of the first absorbent layer. The density of the pledget is 0.14 gm/cc. A single strand of 800 decitex Lycra® elastic is situated beneath the cover layer and adjacent the outer side edges of the first absorbent layer on both of the article's opposed sides. The elastic side members are centered to the absorbents and are approximately 86 mm apart from one another. The elastic side members are approximately 50 mm long and if stretched, doubles its original size prior to placement.

The first absorbent layer and the pledget are enclosed between a 1 mm thick baffle layer of 25 gm/m² polyethylene film and the cover layer by spraying the interior surface of the baffle layer with 10 gm/m² hot melt construction adhesive. A pair of 15 mm wide parallel lines of garment adhesive, 190 mm long with a 10 mm space between them, are applied to the exterior of the baffle and centered with respect the absorbent materials. The garment attachment adhesive is a 50 gm/m² film coating of hot melt. A 50 mm wide by 200 mm long silicon-coated paper is applied to cover the adhesive, with the silicon side toward the adhesive. The baffle and cover layer are cut to a shape which allows a peripheral seal surrounding the absorbent material. The final dimensions of the racetrack-shaped article are 94 mm wide and 238 mm long.

### EXAMPLE 2

The construction of an article according to the present invention is described from the top (or body-side) of the article, progressing downwards. A light amount of hot melt construction adhesive is sprayed beneath the 0.6 oz/yd² spunbond nonwoven cover layer. A first absorbent layer of 150 gm/m² stabilized airlaid with 90% cellulose and 10% binder fiber is cut to a racetrack shape and placed onto the interior of the liner. The density of this airlaid layer is 0.1 gm/cc. This layer is 56 mm wide and 190 mm long. An absorbent pledget of 100% unstabilized fiberized fluff at 650 gm/m² is cut to 36 mm wide by 82 mm long racetrack shape and located beneath the center of the first absorbent layer. The density of the pledget is 0.14 gm/cc. The first absorbent layer and the pledget are enclosed between a fluid impermeable barrier made of 1 mm thick, 25 gm/m² polyethylene film, and the cover layer by spraying both surfaces of the barrier with 10 gm/m² hot melt construction adhesive. An outer ring layer is cut from a 250 gm/m² layer of unstabilized fiberized fluff constructed of 100% cellulose compressed to a density of 0.17 gm/cc. The outer dimensions of this structural layer are 66 mm wide at the crotch, 76 mm wide at the lobes and 218 mm long. A racetrack shaped cavity of 48 mm wide and 170 mm long is cut from the inner part of the structural fluff layer to form a ring and centered to the pledget and upper absorbent layer. A single strand of 800 decitex Lycra® elastic is situated beneath the cover layer and adjacent the outer side edges of the outer ring layer on both of the article's opposed sides. The elastic side members are centered to the absorbents and are approximately 86 mm apart from one another. The elastic side members are approximately 50 mm long and if stretched, doubles its original size prior to placement.

The outer ring is enclosed between a baffle layer of 1 mm thick 25 gm/m² polyethylene film and the fluid impermeable barrier by spraying the interior surface of the baffle with 10 gm/m² hot melt construction adhesive. A pair of 15 mm wide parallel lines of garment adhesive, 190 mm long with a 10 mm space between them, are applied to the exterior of the baffle and centered with respect the absorbent materials. The garment attachment adhesive is a 50 gm/m² film coating of hot melt. A 50 mm wide by 200 mm long silicon-coated paper is applied to cover the adhesive with the silicon side toward the adhesive. The baffle, barrier and the cover layer are cut to a shape which allows a peripheral seal surrounding the absorbent material. The final dimensions of the racetrack-shaped article are 94 mm wide and 238 mm long.

### EXAMPLE 3

The construction of an article according to the present invention is described from the top (or body-side) of the article, progressing downwards. A light amount of hot melt construction adhesive is sprayed beneath the 0.6 oz/yd² spunbond nonwoven cover layer. A first absorbent layer of 150 gm/m² stabilized airlaid with 90% cellulose and 10% binder fiber is cut to a 56 mm wide by 190 mm long racetrack shape and placed onto the interior of the cover layer. The density of this airlaid layer is 0.1 gm/cc. A 100 mm wide layer of 175 gm/m² airlaid pledget of 90% cellulose and 10% binder fiber is folded to an ‘e’ shaped cross-sectional configuration at 36 mm wide and cut at the ends only to a 82 mm long racetrack shape. This pledget airlaid material is 0.1 gm/cc density and located beneath the center of the upper absorbent layer. A second absorbent layer of 125 gm/m² airlaid which is cut in the shape of a dog bone, 66 mm wide at the center-crotch, 76 mm wide at the end portions, and 218 mm long, is located beneath the pledget and centered with regard to the other absorbent materials. This second absorbent layer is 90% cellulose and 10% binder fiber and 0.1 gm/cc. A single strand of 800 decitex Lycra® elastic is situated beneath the cover layer and adjacent the outer side edges of the first absorbent layer on both of the article's opposed sides. The elastic side members are centered to the absorbents and are approximately 86 mm apart from one another. The elastic side members are approximately 50 mm long and if stretched, doubles its original size before placement.

The interior surface of a baffle layer of 1 mm thick 25 gm/m² polyethylene film is sprayed with 10 gm/m² hot melt construction adhesive and the first absorbent layer, the absorbent pledget, and second absorbent layer, are enclosed between the baffle and cover layer. A pair of 15 mm wide parallel lines of garment adhesive, 190 mm long with a 10 mm space between them, are applied to the exterior of the baffle and centered with respect the absorbent materials. The garment attachment adhesive is a 50 gm/m² film coating of hot melt. A 50 mm wide by 200 mm long silicon-coated paper is applied to cover the adhesive with the silicon side toward the adhesive. The baffle and cover layer are cut to a shape which allows a peripheral seal surrounding the absorbent material. The final dimensions of the racetrack-shaped article are 94 mm wide and 238 mm long

TABLE I

| | Areas | | | Center Calculations | | | Surrounding Calculations | | | | |
|---|---|---|---|---|---|---|---|---|---|---|---|
| | | | Surrounding | Center | Basis | | Abs. Weight | Basis | | | |
| | Total | Center | Abs. Area | Abs. | Weight of | Mass | of | Weight | Mass | Basis | |
| | bs. Area | Area | (m²) | Weight | Center | Proportion | Surrounding | of Surr. | Proportion | Weight | Conc. |
| | (m²) | (m²) | (Total − Ctr) | (gm) | (gm/m²) | of Center | (gm) | (gm/m²) | of Center | Ratio | Profile |
| | Aₜ | A_{c} | Aₛ | W_{c} | BW_{c} | MP_{c} | Wₛ | BWₛ | MPₛ | (Ctr/Surr) | CP |
| Conventional Thick Maxi | | | | | | | | | | | |
| pads | | | | | | | | | | | |
| Kotex maxi | 0.0132 | 0.0025 | 0.0107 | 2.9 | 1157 | 455701 | 6.3 | 590 | 55352 | 2.0 | 8.2 |
| Always Maxi | 0.0137 | 0.0025 | 0.0111 | 1.5 | 571 | 224750 | 4.8 | 428 | 38483 | 1.3 | 5.8 |
| Stayfree maxi | 0.0142 | 0.0025 | 0.0117 | 2.1 | 827 | 325501 | 5.4 | 460 | 39425 | 1.8 | 8.3 |
| Kotex maxi (Malaysia) | 0.0132 | 0.0025 | 0.0106 | 1.9 | 740 | 291401 | 5.1 | 480 | 45219 | 1.5 | 6.4 |
| Kotex Curved Maxi | 0.0151 | 0.0025 | 0.0126 | 2.5 | 984 | 387501 | 7.3 | 581 | 46275 | 1.7 | 8.4 |
| Always Curved Maxi | 0.0132 | 0.0025 | 0.0107 | 3.4 | 1327 | 522351 | 4.9 | 460 | 43120 | 2.9 | 12.1 |
| Poise Ultra | 0.0214 | 0.0025 | 0.0188 | 4.4 | 1740 | 685101 | 21.3 | 1133 | 60154 | 1.5 | 11.4 |
| Plus (Inco pad) | | | | | | | | | | | |
| SCA Serenity Ultra (Inco | 0.0189 | 0.0025 | 0.0164 | 4.4 | 1717 | 675801 | 21.0 | 1284 | 78461 | 1.3 | 8.6 |
| pad) | | | | | | | | | | | |
| Ultrathin pads | | | | | | | | | | | |
| Kotex Ultrathin Maxi | 0.0140 | 0.0025 | 0.0114 | 1.3 | 512 | 201500 | 4.0 | 350 | 30591 | 1.5 | 6.6 |
| Always Ultrathin | 0.0156 | 0.0025 | 0.0131 | 1.5 | 571 | 224750 | 4.8 | 364 | 27849 | 1.6 | 8.1 |
| Stayfree Ultrathin | 0.0155 | 0.0025 | 0.0129 | 1.0 | 394 | 155000 | 3.2 | 243 | 18812 | 1.6 | 8.2 |
| Japan (Shiseido) | 0.0116 | 0.0025 | 0.0090 | 1.5 | 587 | 230950 | 1.0 | 109 | 12019 | 5.4 | 19.2 |
| Ultrathin | | | | | | | | | | | |
| Prototypes | | | | | | | | | | | |
| Example 1 | 0.0144 | 0.0025 | 0.0119 | 2.0 | 803 | 316201 | 2.3 | 193 | 16280 | 4.2 | 19.4 |
| Example 2 | 0.0106 | 0.0025 | 0.0081 | 2.0 | 780 | 306901 | 1.3 | 166 | 20627 | 4.7 | 14.9 |
| Example 3 | 0.0155 | 0.0025 | 0.0129 | 2.1 | 815 | 320851 | 3.08 | 238 | 18394 | 3.4 | 17.4 |

The traditional ultrathin article would not function properly if elastic were added to the sides of the article. The traditional ultrathin article's absorbent layers are not strong enough to withstand the tension created by the elastic. This is true of the Shiseido article described in Table I. The Japan (Shiseido) Ultrathin has a concentration profile of 19.4, but it does not contain elastic side members. Furthermore, if elastic side members were to be added to this article, it would collapse longitudinally. Its absorbent layer is merely of tissue-like consistency and does not counteract the tension created by the elastic.

Furthermore, the Shiseido article's basis weight of the region surrounding the center absorbent region (BWₛ) is only 109 gm/m². Therefore, this article does not possess the current advantages of the claimed invention.

While the claimed invention has been described in conjunction with several specific embodiments, it is to be understood that that many alternatives, modifications and variations will be apparent to those skilled in the art in light of the aforegoing description. Accordingly, this invention is intended to embrace all such alternatives, modifications and variations that fall within the spirit and scope of the appended claims.

What is claimed is:

1. A thin absorbent article comprising:
a) a fluid permeable cover layer;
b) a fluid impermeable baffle layer;
c) an absorbent core disposed between the cover layer and the baffle layer, and having a concentration profile of about 13 to about 24, the absorbent core comprising:
i) at least one absorbent layer, and
ii) an absorbent pledget adjacent the at least one absorbent layer; and
d) elastic side members disposed adjacent the baffle layer in at least a portion of opposing side edges of the absorbent article.

2. The absorbent article of claim 1, wherein the cover layer, the fluid impermeable baffle layer, and the at least one absorbent layer have a total thickness from about 1 mm to about 5 mm.

3. The absorbent article of claim 2 wherein the cover layer has a thickness of about 0.1 mm to about 4.0 mm, the baffle layer has a thickness of about 0.1 mm to about 4.0 mm, and the at least one absorbent layer has a thickness of about 0.1 mm to about 4.0 mm.

4. The absorbent article of claim 1, wherein the concentration profile of the article counteracts the tension created by the elastic side members, thereby keeping the article from longitudinally collapsing, and wherein the basis weight of the surrounding region is between about 130 g/m² to about 400 g/m².

5. The absorbent article of claim 1, wherein the absorbent core has a basis weight ratio greater than 3.

6. The absorbent article of claim 1 wherein the absorbent pledget has a length and a width that is less than a length and a width of the at least one absorbent layer.

7. The absorbent article of claim 1, wherein the at least one absorbent layer has a length that is at least 80% of a length of the absorbent article, and the absorbent pledget has a length of about one-third of the length of the first absorbent layer and a width that is less than a width of the first absorbent layer.

8. The absorbent article of claim 1 further comprising an intake layer disposed between the cover layer and the baffle layer.

9. The absorbent article of claim 1 wherein the cover layer is made of apertured material.

10. The absorbent article of claim 1 wherein the absorbent pledget is a superabsorbent material.

11. The absorbent article of claim 1 wherein the absorbent pledget includes odor controlling material.

12. A thin absorbent article comprising:
a) a fluid permeable cover layer;
b) a fluid impermeable baffle layer;
c) an absorbent core disposed between the cover layer and the baffle layer, and having a concentration profile of about 13 to about 24, the absorbent core comprising:
i) a first absorbent layer;
ii) an absorbent pledget;
iii) an outer ring having a central cavity, wherein the absorbent pledget is disposed between the first absorbent layer and the outer ring layer;
d) elastic side members disposed adjacent the baffle layer in at least a portion of opposing side edges of the absorbent article.

13. The absorbent article of claim 12, wherein the fluid permeable cover layer, the fluid impermeable baffle layer, the first absorbent layer, and the outer ring layer, have a total thickness from about 0.1 mm to about 5 mm.

14. The absorbent article of claim 12 wherein the cover layer has a thickness of about 0.1 mm to about 4.0 mm, the baffle layer has a thickness of about 0.1 mm to about 4.0 mm, the first absorbent layer has a thickness of about 0.1 mm to about 4.0 mm, and the outer ring layer has a thickness of about 0.1 mm to about 4.0 mm.

15. The absorbent article of claim 12, wherein the concentration profile of the article counteracts the tension created by the elastic side members, thereby keeping the article from longitudinally collapsing, and wherein the basis weight of the surrounding region is between about 130 g/m² to about 400 g/m².

16. The absorbent article of claim 12, wherein the absorbent core has a basis weight ratio greater than 3.

17. The absorbent article of claim 12, wherein the article further comprises a fluid impermeable barrier layer disposed between the absorbent pledget and the outer ring layer.

18. The absorbent article of claim 12 wherein the central absorbent pledget is disposed within the central cavity of the outer ring layer.

19. The absorbent article of claim 12, wherein the outer ring has a length that is at least 80% of a length of the absorbent article, and the central absorbent pledget having a length of about one-third of the length of the first absorbent layer and a width that is less than a width of the first absorbent layer.

20. The absorbent article of claim 12 wherein the absorbent pledget is a superabsorbent material.

21. The absorbent article of claim 12 wherein the absorbent pledget includes odor controlling material.

22. A thin absorbent article comprising:
a) a liquid permeable cover layer;
b) a liquid impermeable baffle layer;
c) an absorbent core disposed between the cover layer and the baffle layer, and having a concentration profile of about 13 to about 24, the absorbent core comprising:
i) two absorbent layers, and
ii) a central absorbent pledget disposed between the two absorbent layers; and
d) elastic side members disposed adjacent the baffle layer in at least a portion of opposing side edges of the absorbent article.

23. The absorbent article of claim 22 wherein the fluid permeable cover layer, the fluid impermeable baffle layer, the two absorbent layers, have a total thickness from about 0.1 mm to about 5 mm.

24. The absorbent article of claim 22, wherein the concentration profile of the article provides support for tension created by the elastic side members, thereby keeping the article from longitudinally collapsing, and wherein the basis weight of the surrounding region is between about 130 g/m² to about 400 g/m².

25. The absorbent article of claim 22, wherein the absorbent core has a basis weight ratio greater than 3.

26. The absorbent article of claim 22, wherein the two absorbent layers have lengths that are at least 60% of a length of the absorbent article, and the central absorbent pledget having a length of about one-third of the length one of the two absorbent layers and a width that is less than a width of one of the two absorbent layers.

27. The absorbent article of claim 22 wherein the two absorbent layers and the absorbent pledget are made of an airlaid material.

28. The absorbent article of claim 22 wherein the absorbent pledget is comprised of fluff.

29. The absorbent article of claim 22 wherein the absorbent pledget comprises a folded resilient material.

30. The absorbent article of claim 22 wherein the absorbent pledget is folded to have a cross-section in the shape of the letter “e”.

31. The absorbent article of claim 22 wherein the cover layer is made of apertured material.

32. The absorbent article of claim 22 wherein the absorbent pledget is a superabsorbent material.

33. The absorbent article of claim 22 wherein the absorbent pledget includes odor controlling material.

34. A method for preventing the longitudinal collapse of an ultrathin absorbent article fitted with elastic side members comprising providing an absorbent core that has concentration profile of about 13 to about 24.

35. An elasticized absorbent article comprising:
a central absorbent pledget for supporting the shape of the elasticized article, wherein the article has a concentration profile of about 13 to about 24 and does not fold longitudinally onto itself.

36. A method for preventing the longitudinal collapse of an ultrathin absorbent article including a fluid permeable cover layer, a fluid impermeable baffle layer, and elastic side members disposed adjacent the baffle layer in at least a portion of opposing side edges of the absorbent article, comprising providing:
a) an absorbent core disposed between the cover layer and the baffle layer, and having a concentration profile from about 13 to about 24, the absorbent core comprising:
i) at least one absorbent layer, and
ii) an absorbent pledget adjacent the at least one absorbent layer; and
b) wherein a total thickness of each of the fluid permeable cover layer, the fluid impermeable baffle layer, and the at least one absorbent layer is from about 1 mm to about 5 mm.
